(19) United States
(12) Patent Application Publication
(10) Pub. No.: US 2007/0092474 A1
(43) Pub. Date: Apr. 26, 2007

(54) Carnitine salt, preliposome which contains it and dermocosmetic formula for topical use based upon said carnitine salt

(76) Inventor: Giovanni Razzano, Fino Mornasco (IT)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/559,973

(22) PCT Filed: Feb. 17, 2004
(86) PCT No.: PCT/EP04/01471

§ 371 Date: Dec. 7, 2005

(30) Foreign Application Priority Data
Feb. 21, 2003 (IT) MI2003A000316

(51) Int. Cl. A61K 8/42; A61K 9/127; A61K 31/205; C07C 229/02

(52) U.S. Cl. 424/70.21; 514/554; 424/450; 562/553

(57) ABSTRACT

The carnitine salt consisting of carnitine hydroxy citrate of formula (I) of natural origin for treating skin blemishes linked to physiological disorders ascribable to cellulite and anomalous adiposity connected to this. With respect to conventional solutions, the new carnitine salt of the invention proves to be particularly efficient and, above all in dermo-cosmetics, it offers the advantage of having more enhanced permanence and greater efficiency.

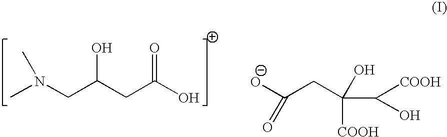

The present invention concerns a new carnitine salt, the preliposome which contains this salt and relative formula for topical use, particularly suitable for dermo-cosmetic applications.

In the field of skin blemishes linked to physiological disorders ascribable to cellulite and the connected adiposity, so-called cellulite identifies a skin pathology that can be attributed to alterations of the lipid metabolic processes, in the presence of subcutaneous lymphatic stases. The convergence of the two disorders leads to the formation of hydrolipid accumulations which, recognised as foreign bodies, trigger a psuedoepithelial coating process thereof. Such a coating in time tends to acquire a rigidity such as to alter the skin surface appearance (orange-peel skin) in the presence of feelings of discomfort to the touch, which in the worst cases are feelings of pain.

Up to now the treatment of this scleropathy, especially in its most full-blown forms, has a surgical solution in liposuction, aimed at emptying the accumulation vesicles. These vesicles, however, have a specific anatomic localisation on the gluteus muscles and on the thighs even if, in obsese subjects, there are different locations (for example the arms). Since pathological aetiology can be attributed to estrogens, the subjects affected are essentially female.

To solve the problems linked to the aforementioned disorders it is known to make use of cosmetic treatments based upon different active ingredients (for example caffeine or natural extracts of marine algae), which both permeating poorly and are not very or limitedly effective. Indeed, the active ingredients currently in use carry out an indirect effect and, for example the iodine contained in the marine algae derivatives, must necessarily be organic to allow it to develop its action mechanism. From this derives the poor efficiency of the product, which translates into a long-lasting action mechanism.

Moreover, the aforementioned known molecules have low permanence (i.e. they are difficult to absorb at the dermal level), with the consequent marked reduction in bioavailability of the active ingredient.

The main purpose of the present invention is that of providing a new active ingredient, particularly suitable for treating skin blemishes linked to physiological disorders identifiable as cellulite and adiposity connected to it.

In particular, a purpose of the invention is that of providing an active ingredient of the aforementioned type which, in relation to those currently known, has improved efficiency and more enhanced permanence.

These and other purposes are accomplished with the new carnitine salt, the preliposome and the dermo-cosmetic formula according to claims 1, 4 and 7 respectively. Preferred ways for realising the invention are given by the remaining claims.

With respect to conventional solutions, the new carnitine salt of the invention proves particularly effective and, above all in dermo-cosmetics, it offers the advantage of having a more enhanced permanence and greater efficiency.

These purposes and advantages are accomplished with the new carnitine salt according to the present invention, consisting of carnitine hydroxycitrate of formula 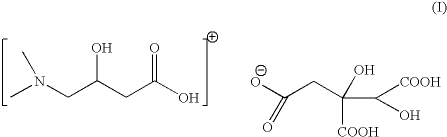
obtained from the reaction of 3-carboxy-2-hydroxy-propyl trimethyl amine (commercial name “carnitine) of formula 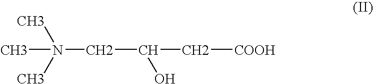
with L-hydroxy citric acid of the formula

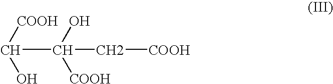

In particular, L-hydroxy citric acid of formula (III) is that which is present in the juices of fruits of the botanical species known as Garcinia Cambogia, originating from South-east Asia, also known as Tamarindo Malabar.

Despite the renown of the antilipidimising effect of the reactants of the new carnitine salt of the invention, the greater efficiency and the improved permanence of carnitine hydroxy citrate are the result of an absolutely unexpected and surprising synergic effect.

Hereafter the preferred method for preparing the new carnitine salt of the invention shall be described.

### PREPARATION OF CARNITINE HYDROXY CITRATE

The hydroxy citric acid reactant was prepared starting from the calcium salt which is the derivative of direct extraction from the juice of Garcinia Cambogia fruits. Calcic salification is necessary to stabilise the acid and to obtain a substance that is easy to manipulate in practical use of formulas with supplementary dietetic action.

1040 grams of calcium hydroxy citrate at 75% (3.14 mols) thus prepared are dissolved in 1500 ml of purified water. 395 grams of dihydrate oxalic acid are added to the solution (3.14 mols). A voluminous precipitate of calcium oxylate is obtained, able to be filtered with some difficulty (it is better to centrifuge). 497 grams of base carnitine (3.08 mols) are added to the limpid, straw-coloured hydroxy citric acid solution, taking them to complete solubilisation. The solution has a content equal to 43% carnitine hydroxy citrate and a pH of 4.45.

With the carnitine hydroxy citrate obtained with the previous method, the preliposome and the formula for dermatological use are prepared according to the following examples.

### PREPARATION OF THE PRELIPOSOME

With the help of a Silverson disperser in 2143 grams of the described carnitine hydroxy citrate solution, in cold state, 535.75 grams of pure phosphatidylcholine are dispersed. With dispersion completed 42.86 grams of xantana rubber are added for optimal stabilisation of the viscosity of the preparation.

### PREPARATION OF THE COSMETIC GEL

The preliposome described above is a preparation capable of producing liposomial vesicles, containing the active ingredient, at the time of its dispersion in an aqueous means free from surface-active agents. To carry out the activity test a preparation in gel form was used the composition of which corresponded to the formula indicated hereafter:

| | | |
|---|---|---|
| Hydroxy ethyl cellulose | 1.8 | g |
| Triethanol amine | 0.9 | g |
| Solubiliser, fragrance, preservatives | 2.5 | g |
| Preliposome | 13 | g |
| Purified water | q.s. 100 | g |

The preparation is obtained through the use of a turboemulsifier in vacuo and the characteristics of the product corresponded to the following chemical physical parameters: pH=4.70.

The improved efficiency of the carnitine salt of the invention was proven with the following dermatological tests.

### DERMATOLOGICAL TESTS

A dermatological test was carried out on 25 female volunteers between 18 and 65 years of age, with the backing of the Department of Physiological and Pharmacological Sciences of the university of Pavia. The investigation aimed to check the modification of three anatomical-physiological parameters, qualifying the unaesthetic-functional aspect connected to the manifestation of so-called cellulite and subcutaneous hypertriglyceridaemia: measurement of the skin folds, measurement of the circumference of the thighs, thermographic skin measurement.

The test stretched out over 60 days with the continual daily application of the aforementioned preparation of cosmetic gel. The measurements were carried out after 15, 30 and 60 days. The detail of the test protocol and of the results obtained is shown hereafter.

Evaluation of the Efficiency of a Cosmetic Product Adjuvating the Reduction of the Skin Blemishes Deriving from Cellulite and from Localised Adiposity.

Purpose

The present experimentation allows it to be evaluated whether the cosmetic product being studied carries out an action adjuvating the reduction of the skin blemishes deriving from cellulite and from localised adiposity.

Volunteers Taking Part in the Study

25 female volunteers aged between 18 and 65, with the presence of skin blemishes caused by cellulite and localised adiposity, were selected according to the following criteria: good general state of health, absence of skin pathologies, not being under medication, commitment not to vary the normal daily routine, no medical history of atopy.

Preparation of the Samples

The samples were applied according to the indications supplied by the Client.

Method of Application of the Samples

The samples were tested by uniformly applying them on “Glute SX Glute DJ (Femoris Posto SX Femoris Posto DX Femoris Ant. SX Femoris Ant. DX” every day at least once per day for the entire duration of the test.

Carrying Out the Test

At the time of inclusion in the study and during the subsequent checks after 15 days (T15), after 30 days (T30) and after 60 days (T60) of treatment the following instrumental measurements were taken:
plicometry (through a plicometer)
thigh circumference (through a measuring tape broken into millimetres)
contact thermography (through liquid crystal thermographic plates).

In particular, the plicometer used in the test constitutes the suitable instrument for measuring the skin plica thickness. Skin plicometry provides numerical data of particular use when the panniculopathic process of the cellulite is associated with localised adiposity.

For its part, thermography allows the extension and the gravity of the microcirculatory skin damage and, consequently, the subcutaneous damage, to be evaluated based upon the regional skin temperature variations. Such temperature variations reveal the presence of subschemic areas (cold areas, due to regional hypothermia) or of phenomena of capillary-venous stasis (hot areas). The diagnostic properties of thermography are relative to the recognition of maldistribution of microcirculation. In particular, “liquid crystal contact thermography” was used, in which the property of cholesteric liquid crystals (of which the plates are made up) of modifying their spatial arrangement according to the temperature is exploited; each spatial modification corresponds to a different refraction of the light rays which thus assume different colours according to the incidence of the light on the faces of the cholesteric microcrystals: in practice, when the thermographic plates are placed upon the skin of the zone to be investigated, these assume different colourations according to the skin temperature. Conventionally, the thermographic images observed during the course of the different stages of evolution of the Edematous fibrosclerotic panniculopathy from stasis of the lower limbs can be classified in four, or more, thermographic stages; the latter approximately correspond to the gravity of the four istologic stages in which the moments of evolution of the panniculosis are divided.

Evaluation and Calculation of the Results

The images displayed on the thermographic plate (attached) require computer reprocessing to allow there to be an arbitrary point score assigned to them associated with the stage and gravity of the cellulite. The clinical score used for thermographic analysis is shown in the following table:

| CELLULITE STUDY | CLINICAL SCORE |
|---|---|
| IV SERIOUS | 9 |
| IV AVERAGE | 8 |
| IV INITIAL | 7 |
| III SERIOUS | 6 |
| III AVERAGE | 5 |
| III INITIAL | 4 |
| II SERIOUS | 3 |
| II AVERAGE | 2 |
| II INITIAL | 1 |

The results of the tests are gathered in the attached tables.

### CONCLUSIONS

Based upon the results obtained, according to the methods used, it can be stated that the product under examination is able to substantially modify, in the period of treatment, the clinical parameters evaluated on the volunteers subjected to tests. The treatment proved able to adjuvate the reduction in skin blemishes deriving from cellulite and the reduction of localised adiposity.

Comparatively, the same previous tests that were carried out in carnitine hydroxy citrate were repeated for just carnitine of formula (II) and just hydroxy citric acid of formula (III), obtaining the following average values:

| TEST | |
|---|---|
| PLICOMETRY DX | PLICOMETRY SX |

| | | | Hydroxy | | | | Hydroxy | |
|---|---|---|---|---|---|---|---|---|
| Active | Carnitine | | citric | | | | citric | |
| ingredient | (II) | | acid | | Carnitine | | acid | |
| Average | 25.7 | 23.7 | 30.9 | 29.0 | 25.7 | 23.7 | 30.9 | 29.9 |
| values | | | | | | | | |

From a comparison with the corresponding tables relative to the two reactants by themselves, carnitine hydroxy citrate demonstrates that it provides the best results; due to the greater efficiency and the increased permanence of the salt with respect to the starting reactants.

Carnitine hydroxy citrate salt, which in the previous carnitine hydroxy citrate examples was used for dermatological treatments, can in reality also have different uses, for example as a food supplement and the like, all of which are covered by the present invention.

[image]

TABLE 1
RESULTS

| | RIGHT PLICOMETRY | | | | | | | | | | | |
|---|---|---|---|---|---|---|---|---|---|---|---|---|
| Volunteer | 1/3 | | | | 2/3 | | | | 3/3 | | | |
| No. | T0 | T15 | T30 | T60 | T0 | T15 | T30 | T60 | T0 | T15 | T30 | T60 |
| 01MT | 22.8 | 21.4 | 20.5 | 19.9 | 28.8 | 27.3 | 26.2 | 25.6 | 22.9 | 21.9 | 20.4 | 19.7 |
| 02LT | 34.6 | 33.2 | 32.5 | 31.7 | 39.8 | 37.3 | 35.6 | 35.3 | 36.2 | 34.9 | 34.0 | 33.2 |
| 03MA | 16.8 | 15.4 | 14.5 | 13.8 | 27.0 | 27.0 | 24.3 | 23.8 | 26.8 | 25.8 | 25.6 | 23.7 |
| 04ME | 30.5 | 29.0 | 28.2 | 27.5 | 37.2 | 35.7 | 34.6 | 34.0 | 40.1 | 40.0 | 40.2 | 38.9 |
| 05CM | 23.3 | 22.0 | 18.9 | 20.5 | 30.8 | 28.5 | 28.2 | 26.3 | 41.6 | 40.0 | 39.9 | 37.4 |
| 06FE | 17.8 | 16.5 | 15.5 | 15.0 | 26.7 | 25.2 | 24.0 | 23.2 | 25.9 | 24.9 | 23.6 | 22.3 |
| 07FA | 35.9 | 34.5 | 33.2 | 32.1 | 32.8 | 30.8 | 30.2 | 29.4 | 32.7 | 31.7 | 30.4 | 29.4 |
| 08BA | 24.9 | 23.5 | 22.4 | 22.0 | 20.9 | 19.4 | 18.2 | 17.3 | 29.9 | 28.9 | 27.6 | 27.0 |
| 09LE | 21.1 | 19.6 | 18.8 | 17.8 | 29.2 | 27.7 | 25.4 | 26.0 | 33.8 | 32.8 | 31.5 | 32.1 |
| 10BB | 25.9 | 24.5 | 23.6 | 23.0 | 36.8 | 35.3 | 34.0 | 32.8 | 37.1 | 37.5 | 36.9 | 35.2 |
| 11RD | 19.8 | 18.4 | 17.5 | 16.7 | 26.3 | 24.8 | 23.7 | 23.5 | 39.8 | 39.6 | 38.4 | 36.8 |
| 12MZ | 26.7 | 25.3 | 24.4 | 23.7 | 34.9 | 33.4 | 33.5 | 33.6 | 40.6 | 39.8 | 38.5 | 37.4 |
| 13OP | 26.0 | 24.2 | 24.2 | 24.6 | 35.5 | 34.0 | 32.9 | 32.1 | 29.9 | 28.9 | 27.6 | 26.9 |
| 14DT | 31.1 | 30.0 | 28.8 | 27.6 | 33.5 | 32.0 | 30.0 | 30.0 | 37.7 | 37.8 | 36.2 | 35.6 |
| 15CS | 26.6 | 27.1 | 26.5 | 26.6 | 29.7 | 27.3 | 27.6 | 26.5 | 36.8 | 35.9 | 33.6 | 33.2 |
| 16LM | 19.7 | 19.5 | 17.4 | 16.7 | 24.8 | 23.0 | 21.7 | 20.9 | 28.8 | 28.2 | 26.3 | 25.3 |
| 17EFC | 22.9 | 23.0 | 22.7 | 21.5 | 29.9 | 29.6 | 26.5 | 26.7 | 29.4 | 28.4 | 26.5 | 26.6 |
| 18CCO | 24.6 | 23.2 | 21.8 | 21.7 | 22.4 | 20.9 | 20.0 | 20.0 | 25.6 | 26.1 | 23.7 | 22.3 |
| 19EP | 32.8 | 31.0 | 31.2 | 28.9 | 31.2 | 31.5 | 30.8 | 27.8 | 32.3 | 32.0 | 31.2 | 29.5 |
| 20AMA | 24.9 | 23.5 | 22.6 | 21.4 | 33.7 | 32.2 | 31.0 | 29.7 | 39.9 | 38.9 | 37.6 | 35.4 |
| 21PL | 22.9 | 20.3 | 20.5 | 18.4 | 27.7 | 27.8 | 25.4 | 25.6 | 22.3 | 21.3 | 19.6 | 18.2 |
| 22CA | 28.1 | 26.7 | 25.8 | 24.1 | 26.8 | 25.3 | 23.9 | 23.9 | 36.8 | 37.0 | 36.4 | 34.1 |
| 23BTC | 24.0 | 22.6 | 21.7 | 21.3 | 25.3 | 24.8 | 22.3 | 21.8 | 19.8 | 18.8 | 17.5 | 17.0 |
| 24LB | 30.8 | 29.4 | 27.6 | 28.0 | 37.8 | 38.1 | 38.1 | 37.7 | 35.1 | 34.1 | 30.5 | 30.9 |
| 25GN | 27.9 | 26.5 | 24.3 | 23.9 | 41.5 | 40.0 | 40.0 | 40.0 | 42.6 | 41.6 | 39.8 | 39.8 |
| MEDIA | 25.7 | 24.4 | 23.4 | 22.7 | 30.9 | 29.6 | 28.3 | 27.7 | 33.0 | 32.3 | 30.9 | 29.9 |

RIGHT LEG PLICAS

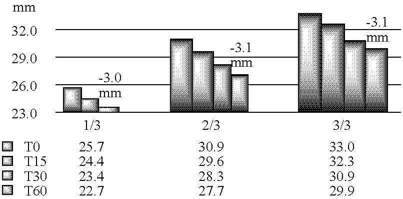

TABLE 2

| | LEFT PLICOMETRY | | | | | | | | | | | |
|---|---|---|---|---|---|---|---|---|---|---|---|---|
| | 1/3 | | | | 2/3 | | | | 3/3 | | | |
| Volunteer | T0 | T15 | T30 | T60 | T0 | T15 | T30 | T60 | T0 | T15 | T30 | T60 |
| 01MT | 22.8 | 22.8 | 21.6 | 21.5 | 25.3 | 24.6 | 23.0 | 22.3 | 22.9 | 22.3 | 20.8 | 19.9 |
| 02LT | 34.6 | 33.9 | 32.3 | 30.4 | 41.6 | 40.9 | 39.3 | 38.6 | 40.9 | 40.3 | 38.8 | 37.9 |
| 03MA | 16.8 | 16.1 | 14.5 | 14.0 | 23.5 | 22.8 | 21.2 | 20.5 | 28.9 | 28.3 | 26.8 | 25.9 |
| 04ME | 30.5 | 29.8 | 28.2 | 27.7 | 32.5 | 31.8 | 30.2 | 29.5 | 38.1 | 37.5 | 36.0 | 35.1 |
| 05CM | 23.3 | 22.6 | 21.0 | 20.5 | 28.7 | 28.0 | 26.4 | 25.7 | 34.9 | 34.3 | 32.8 | 31.9 |
| 06FE | 17.8 | 17.1 | 15.5 | 15.0 | 22.6 | 21.9 | 20.3 | 19.6 | 21.8 | 21.2 | 19.7 | 18.8 |
| 07FA | 35.9 | 35.2 | 33.6 | 30.9 | 38.8 | 38.1 | 36.5 | 35.8 | 37.9 | 37.3 | 35.8 | 34.9 |
| 08BA | 24.9 | 24.2 | 22.6 | 22.1 | 24.8 | 24.1 | 22.5 | 21.8 | 27.8 | 27.2 | 25.7 | 24.8 |
| 09LE | 21.1 | 20.4 | 18.8 | 18.3 | 27.1 | 26.4 | 24.8 | 24.1 | 30.8 | 30.2 | 28.7 | 27.8 |
| 10BB | 25.9 | 25.2 | 23.6 | 23.1 | 28.9 | 28.2 | 26.6 | 25.9 | 33.3 | 32.7 | 31.2 | 30.3 |
| 11RD | 19.8 | 19.1 | 17.5 | 17.0 | 28.1 | 27.4 | 25.8 | 25.1 | 26.8 | 26.2 | 24.7 | 23.8 |
| 12MZ | 26.7 | 26.0 | 24.4 | 23.9 | 40.9 | 40.2 | 38.6 | 37.9 | 41.6 | 41.0 | 39.5 | 38.6 |
| 13OP | 26.0 | 25.3 | 23.7 | 23.2 | 33.9 | 33.2 | 31.6 | 30.9 | 28.1 | 27.5 | 26.0 | 25.1 |
| 14DT | 31.1 | 30.4 | 28.8 | 27.6 | 30.8 | 30.1 | 28.5 | 27.8 | 35.9 | 35.3 | 33.8 | 33.2 |
| 15CS | 26.6 | 27.2 | 24.3 | 23.6 | 27.6 | 26.9 | 25.3 | 24.6 | 35.3 | 34.7 | 33.2 | 32.3 |
| 16LM | 19.7 | 19.6 | 17.4 | 16.5 | 24.3 | 23.6 | 22.0 | 21.3 | 20.6 | 20.0 | 18.5 | 17.6 |
| 17EFC | 22.9 | 22.6 | 20.6 | 18.4 | 27.8 | 27.1 | 25.5 | 24.8 | 33.5 | 32.9 | 31.4 | 29.6 |
| 18CCO | 24.6 | 23.9 | 22.3 | 21.2 | 20.6 | 19.9 | 18.3 | 17.6 | 20.1 | 19.5 | 18.5 | 17.2 |
| 19EP | 32.8 | 32.9 | 30.5 | 30.5 | 31.8 | 31.1 | 29.5 | 28.8 | 33.5 | 32.9 | 31.4 | 30.5 |
| 20AMA | 24.9 | 24.2 | 23.5 | 22.1 | 29.9 | 29.2 | 27.6 | 26.9 | 35.9 | 35.3 | 33.8 | 32.9 |
| 21PL | 22.9 | 22.6 | 20.2 | 19.8 | 22.9 | 22.2 | 20.6 | 19.9 | 20.3 | 19.7 | 18.2 | 18.5 |
| 22CA | 28.1 | 27.4 | 25.8 | 25.3 | 27.2 | 26.5 | 24.9 | 24.2 | 37.2 | 36.6 | 35.1 | 34.2 |
| 23BTC | 24.0 | 24.6 | 23.8 | 20.7 | 25.4 | 24.7 | 23.0 | 22.4 | 17.8 | 17.2 | 15.7 | 14.8 |
| 24LB | 30.8 | 30.6 | 28.5 | 26.2 | 35.4 | 34.7 | 33.5 | 32.4 | 34.1 | 33.5 | 32.0 | 31.0 |
| 25GN | 27.9 | 27.8 | 25.6 | 23.9 | 39.7 | 39.0 | 37.4 | 36.7 | 41.5 | 40.9 | 39.4 | 38.5 |
| MEDIA | 25.7 | 25.3 | 23.5 | 22.5 | 29.6 | 28.9 | 27.3 | 26.6 | 31.2 | 30.6 | 29.1 | 28.2 |

LEFT LEG PLICAS

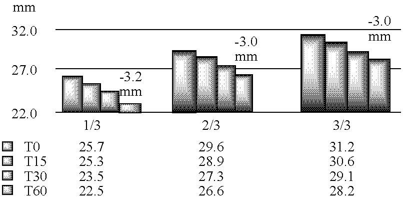

TABLE 3

| | RIGHT THIGH CIRCUMFERENCE | | | | | | | | | | | |
|---|---|---|---|---|---|---|---|---|---|---|---|---|
| Volunteer | 1/3 | | | | 2/3 | | | | 3/3 | | | |
| No. | T0 | T15 | T30 | T60 | T0 | T15 | T30 | T60 | T0 | T15 | T30 | T60 |
| 01MT | 50.8 | 50.4 | 49.0 | 48.3 | 45.3 | 43.2 | 43.0 | 41.7 | 36.4 | 36.4 | 35.7 | 33.7 |
| 02LT | 60.8 | 60.4 | 59.0 | 57.8 | 55.6 | 54.2 | 53.9 | 52.7 | 43.8 | 43.9 | 42.8 | 42.4 |
| 03MA | 48.2 | 48.0 | 46.4 | 45.7 | 45.4 | 43.7 | 44.2 | 43.2 | 36.3 | 35.8 | 35.6 | 34.3 |
| 04ME | 56.0 | 59.8 | 58.4 | 56.5 | 54.6 | 52.9 | 51.3 | 50.8 | 46.4 | 45.5 | 44.3 | 44.0 |
| 05CM | 55.0 | 55.9 | 54.5 | 53.8 | 49.9 | 48.2 | 48.2 | 46.8 | 41.2 | 40.6 | 40.5 | 38.2 |
| 06FE | 53.2 | 52.8 | 51.4 | 50.7 | 51.3 | 49.6 | 48.0 | 48.1 | 41.2 | 40.8 | 40.3 | 39.3 |
| 07FA | 52.0 | 53.2 | 51.8 | 50.0 | 49.0 | 47.3 | 46.8 | 46.5 | 39.7 | 37.7 | 36.6 | 36.2 |
| 08BA | 64.7 | 65.3 | 63.9 | 62.4 | 58.4 | 56.7 | 56.4 | 56.4 | 48.0 | 49.2 | 48.1 | 47.7 |
| 09LE | 55.7 | 55.3 | 53.9 | 53.2 | 52.5 | 50.7 | 50.3 | 49.2 | 37.6 | 38.5 | 38.2 | 37.0 |
| 10BB | 50.6 | 49.4 | 48.0 | 47.3 | 47.2 | 46.2 | 46.5 | 43.7 | 36.4 | 35.3 | 34.2 | 33.8 |
| 11RD | 48.1 | 48.4 | 47.0 | 46.0 | 46.5 | 44.5 | 44.3 | 44.5 | 37.7 | 37.2 | 36.6 | 35.7 |
| 12MZ | 49.0 | 49.8 | 48.4 | 47.2 | 47.9 | 46.2 | 44.6 | 44.7 | 38.4 | 37.1 | 36.4 | 35.6 |
| 13OP | 53.0 | 53.3 | 51.9 | 51.0 | 50.4 | 49.8 | 49.6 | 47.2 | 39.8 | 39.7 | 38.7 | 38.2 |
| 14DT | 56.9 | 57.6 | 56.2 | 55.5 | 52.9 | 52.3 | 51.7 | 49.7 | 37.8 | 38.5 | 38.4 | 37.0 |
| 15CS | 59.0 | 59.2 | 57.8 | 56.7 | 55.9 | 55.6 | 55.4 | 53.8 | 45.3 | 44.5 | 43.4 | 43.0 |
| 16LM | 55.2 | 54.3 | 52.9 | 52.0 | 56.8 | 56.5 | 56.0 | 56.0 | 43.2 | 42.3 | 41.0 | 40.8 |
| 17EFC | 52.6 | 50.5 | 49.3 | 48.4 | 49.2 | 48.7 | 48.6 | 46.3 | 37.4 | 36.6 | 36.2 | 34.8 |
| 18CCO | 47.0 | 48.1 | 46.7 | 44.7 | 46.7 | 45.0 | 45.0 | 43.5 | 38.6 | 37.7 | 36.8 | 36.2 |
| 19EP | 55.7 | 56.3 | 53.6 | 52.8 | 52.6 | 50.9 | 50.0 | 49.4 | 39.0 | 39.2 | 38.7 | 37.7 |
| 20AMA | 47.0 | 48.0 | 47.1 | 45.0 | 44.0 | 44.0 | 44.0 | 43.5 | 37.9 | 35.5 | 35.2 | 34.2 |
| 21PL | 48.3 | 48.8 | 46.5 | 45.9 | 45.4 | 45.2 | 44.6 | 44.3 | 39.5 | 38.7 | 37.8 | 37.2 |
| 22CA | 52.2 | 51.8 | 49.9 | 49.3 | 52.0 | 51.8 | 50.6 | 50.2 | 47.5 | 39.4 | 38.3 | 37.9 |
| 23BTC | 54.9 | 54.5 | 53.0 | 51.8 | 51.4 | 51.0 | 51.0 | 48.3 | 44.1 | 43.2 | 42.5 | 41.7 |
| 24LB | 50.2 | 51.0 | 48.6 | 48.1 | 47.9 | 47.3 | 47.0 | 45.9 | 38.4 | 37.5 | 36.4 | 36.0 |
| 25GN | 52.0 | 50.8 | 48.5 | 48.5 | 48.2 | 47.9 | 47.8 | 45.0 | 37.6 | 37.7 | 36.8 | 36.2 |
| MEDIA | 53.1 | 53.3 | 51.8 | 50.7 | 50.3 | 49.2 | 48.8 | 47.7 | 40.4 | 39.5 | 38.8 | 38.0 |

RIGHT THIGH CIRCUMFERENCE

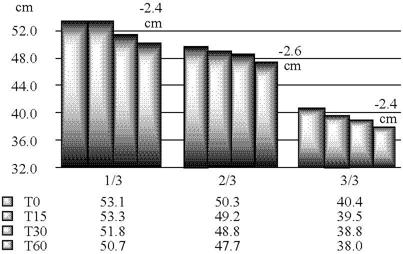

TABLE 4

| | LEFT THIGH CIRCUMFERENCE | | | | | | | | | | | |
|---|---|---|---|---|---|---|---|---|---|---|---|---|
| Volunteer | 1/3 | | | | 2/3 | | | | 3/3 | | | |
| No. | T0 | T15 | T30 | T60 | T0 | T15 | T30 | T60 | T0 | T15 | T30 | T60 |
| 01MT | 49.7 | 49.2 | 48.1 | 47.4 | 44.9 | 44.4 | 43.5 | 42.6 | 36.4 | 35.9 | 34.7 | 34.2 |
| 02LT | 60.6 | 60.5 | 59.0 | 57.9 | 55.9 | 55.4 | 54.5 | 53.6 | 43.8 | 43.3 | 42.1 | 41.6 |
| 03MA | 48.2 | 47.7 | 46.6 | 45.9 | 45.4 | 44.9 | 44.0 | 43.1 | 36.3 | 35.8 | 34.6 | 34.1 |
| 04ME | 59.2 | 58.7 | 57.6 | 56.9 | 54.6 | 54.1 | 53.2 | 52.3 | 46.4 | 45.9 | 44.7 | 44.2 |
| 05CM | 55.0 | 54.5 | 53.4 | 52.7 | 49.9 | 49.4 | 48.5 | 47.6 | 41.2 | 40.7 | 39.5 | 39.0 |
| 06FE | 52.8 | 52.3 | 51.2 | 50.5 | 51.3 | 50.8 | 49.9 | 49.0 | 41.2 | 40.7 | 39.5 | 39.0 |
| 07FA | 51.7 | 51.2 | 50.1 | 49.4 | 49.0 | 48.5 | 47.6 | 46.7 | 39.7 | 39.2 | 38.0 | 37.5 |
| 08BA | 64.0 | 63.5 | 62.4 | 61.7 | 58.4 | 57.9 | 57.0 | 56.1 | 48.0 | 47.5 | 46.3 | 45.8 |
| 09LE | 55.7 | 55.2 | 54.2 | 53.4 | 52.4 | 51.9 | 51.0 | 50.1 | 37.6 | 37.1 | 35.9 | 35.4 |
| 10BB | 51.0 | 50.5 | 49.2 | 48.7 | 46.9 | 46.4 | 45.5 | 44.6 | 36.4 | 35.9 | 34.7 | 34.2 |
| 11RD | 47.6 | 47.1 | 46.0 | 44.9 | 46.2 | 45.7 | 44.8 | 43.9 | 37.7 | 37.2 | 36.0 | 35.5 |
| 12MZ | 49.0 | 48.5 | 47.4 | 46.7 | 47.9 | 47.4 | 46.5 | 45.6 | 38.4 | 37.9 | 36.7 | 36.2 |
| 13OP | 52.5 | 52.0 | 50.9 | 50.0 | 50.4 | 49.9 | 49.0 | 48.1 | 39.8 | 39.3 | 38.1 | 37.6 |
| 14DT | 56.6 | 56.1 | 55.0 | 53.7 | 52.9 | 52.4 | 51.5 | 50.6 | 37.8 | 37.3 | 36.1 | 35.6 |
| 15CS | 58.1 | 57.6 | 56.5 | 55.8 | 55.9 | 55.4 | 54.5 | 53.6 | 43.1 | 42.6 | 41.4 | 41.6 |
| 16LM | 55.2 | 54.7 | 53.6 | 51.7 | 56.7 | 56.2 | 55.3 | 54.4 | 43.2 | 42.7 | 41.5 | 41.0 |
| 17EFC | 52.8 | 52.3 | 51.2 | 50.5 | 48.9 | 48.4 | 47.5 | 46.6 | 37.4 | 36.9 | 35.7 | 35.2 |
| 18CCO | 47.1 | 46.6 | 45.5 | 44.8 | 46.7 | 46.2 | 45.3 | 44.4 | 38.7 | 38.2 | 37.0 | 36.5 |
| 19EP | 55.7 | 55.2 | 54.1 | 52.9 | 52.6 | 52.1 | 51.2 | 50.3 | 38.7 | 38.2 | 37.0 | 36.5 |
| 20AMA | 47.0 | 46.5 | 45.4 | 44.7 | 44.0 | 43.5 | 42.6 | 41.7 | 37.9 | 37.4 | 36.2 | 35.7 |
| 21PL | 48.5 | 48.0 | 46.9 | 46.2 | 45.4 | 44.9 | 44.0 | 43.1 | 39.7 | 39.2 | 38.0 | 37.5 |
| 22CA | 52.1 | 51.6 | 50.5 | 49.8 | 52.0 | 51.5 | 50.6 | 49.7 | 47.2 | 46.7 | 45.5 | 45.0 |
| 23BTC | 55.7 | 55.2 | 54.0 | 53.4 | 51.4 | 50.9 | 50.0 | 49.1 | 44.2 | 43.7 | 43.6 | 42.0 |
| 24LB | 50.2 | 49.7 | 49.7 | 48.5 | 47.9 | 47.4 | 46.5 | 45.6 | 38.6 | 38.1 | 38.1 | 36.4 |
| 25GN | 52.7 | 52.2 | 52.3 | 50.4 | 48.2 | 47.7 | 46.8 | 45.9 | 37.6 | 37.1 | 35.9 | 35.4 |
| MEDIA | 53.1 | 52.7 | 51.6 | 50.7 | 50.2 | 49.7 | 48.8 | 47.9 | 40.3 | 39.8 | 38.7 | 38.1 |

LEFT THIGH CIRCUMFERENCE

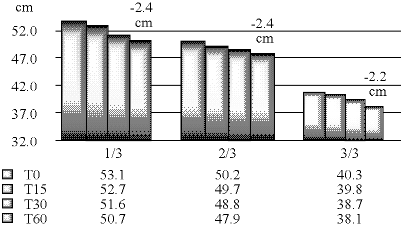

| | TABELLA 5 | | | | | | | | | | | |
|---|---|---|---|---|---|---|---|---|---|---|---|---|
| | RIGHT THIGH THERMOGRAPHY | | | | | | | | | | | |
| Volunteer | 1/3 | | | | 2/3 | | | | 3/3 | | | |
| No. | T0 | T15 | T30 | T60 | T0 | T15 | T30 | T60 | T0 | T15 | T30 | T60 |
| 01MT | 5 | 5 | 4 | 2 | 3 | 2 | 2 | 1 | 2 | 2 | 1 | 1 |
| 02LT | 8 | 7 | 7 | 6 | 6 | 6 | 4 | 4 | 7 | 5 | 4 | 3 |
| 03MA | 6 | 5 | 2 | 2 | 6 | 5 | 3 | 3 | 4 | 3 | 2 | 2 |
| 04ME | 6 | 6 | 5 | 4 | 6 | 6 | 4 | 4 | 4 | 4 | 3 | 2 |
| 05CM | 5 | 5 | 2 | 2 | 4 | 3 | 2 | 2 | 3 | 3 | 2 | 1 |
| 06FE | 5 | 4 | 2 | 2 | 5 | 4 | 2 | 2 | 5 | 3 | 3 | 3 |
| 07FA | 4 | 4 | 2 | 2 | 4 | 4 | 3 | 3 | 4 | 4 | 3 | 2 |
| 08BA | 7 | 7 | 4 | 3 | 7 | 5 | 3 | 3 | 5 | 5 | 4 | 3 |
| 09LE | 3 | 3 | 2 | 2 | 3 | 3 | 3 | 2 | 3 | 2 | 2 | 1 |
| 10BB | 5 | 4 | 3 | 2 | 5 | 4 | 3 | 3 | 4 | 3 | 3 | 3 |
| 11RD | 6 | 6 | 4 | 3 | 5 | 4 | 3 | 3 | 3 | 2 | 2 | 2 |
| 12MZ | 5 | 5 | 4 | 4 | 5 | 5 | 4 | 4 | 4 | 4 | 3 | 3 |
| 13OP | 3 | 3 | 2 | 2 | 4 | 3 | 3 | 2 | 4 | 3 | 2 | 2 |
| 14DT | 5 | 5 | 4 | 3 | 5 | 4 | 3 | 2 | 4 | 4 | 3 | 3 |
| 15CS | 4 | 4 | 4 | 2 | 4 | 3 | 3 | 2 | 4 | 3 | 3 | 2 |
| 16LM | 5 | 5 | 4 | 4 | 5 | 4 | 3 | 3 | 4 | 4 | 3 | 3 |
| 17EFC | 6 | 6 | 5 | 4 | 5 | 5 | 4 | 3 | 4 | 4 | 4 | 3 |
| 18CCO | 7 | 7 | 6 | 5 | 5 | 4 | 4 | 3 | 3 | 2 | 2 | 2 |
| 19EP | 7 | 6 | 6 | 5 | 6 | 6 | 5 | 4 | 4 | 4 | 2 | 2 |
| 20AMA | 5 | 5 | 4 | 3 | 4 | 4 | 4 | 3 | 4 | 3 | 3 | 3 |
| 21PL | 6 | 6 | 5 | 3 | 5 | 4 | 3 | 3 | 4 | 4 | 4 | 3 |
| 22CA | 4 | 4 | 4 | 3 | 4 | 4 | 3 | 3 | 4 | 4 | 3 | 2 |
| 23BTC | 6 | 5 | 5 | 4 | 5 | 4 | 4 | 3 | 4 | 4 | 4 | 3 |
| 24LB | 5 | 4 | 2 | 2 | 5 | 4 | 2 | 2 | 5 | 5 | 4 | 4 |
| 25GN | 5 | 5 | 3 | 2 | 3 | 3 | 3 | 2 | 5 | 4 | 4 | 3 |
| MEDIA | 5.3 | 5.0 | 3.8 | 3.0 | 4.8 | 4.1 | 3.2 | 2.8 | 4.0 | 3.5 | 2.9 | 2.4 |

RIGHT THIGH THERMOGRAPHY

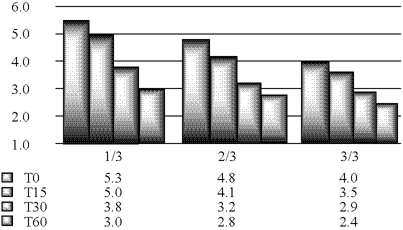

TABLE 6

| | LEFT THIGH THERMOGRAPHY | | | | | | | | | | | |
|---|---|---|---|---|---|---|---|---|---|---|---|---|
| Volunteer | 1/3 | | | | 2/3 | | | | 3/3 | | | |
| No. | T0 | T15 | T30 | T60 | T0 | T15 | T30 | T60 | T0 | T15 | T30 | T60 |
| 01MT | 5 | 5 | 4 | 2 | 3 | 2 | 2 | 1 | 2 | 2 | 1 | 1 |
| 02LT | 7 | 6 | 6 | 6 | 6 | 6 | 4 | 4 | 7 | 5 | 4 | 3 |
| 03MA | 6 | 5 | 2 | 2 | 6 | 5 | 3 | 3 | 4 | 3 | 2 | 2 |
| 04ME | 6 | 6 | 5 | 4 | 6 | 6 | 4 | 4 | 4 | 4 | 3 | 2 |
| 05CM | 5 | 5 | 2 | 2 | 4 | 3 | 2 | 2 | 3 | 3 | 2 | 1 |
| 06FE | 5 | 4 | 2 | 2 | 5 | 4 | 2 | 2 | 5 | 3 | 3 | 3 |
| 07FA | 4 | 3 | 2 | 2 | 3 | 3 | 2 | 2 | 4 | 4 | 3 | 2 |
| 08BA | 7 | 7 | 4 | 3 | 7 | 5 | 3 | 3 | 5 | 5 | 4 | 3 |
| 09LE | 2 | 2 | 2 | 2 | 2 | 3 | 2 | 2 | 2 | 2 | 1 | 1 |
| 10BB | 5 | 4 | 3 | 2 | 5 | 4 | 3 | 3 | 4 | 3 | 3 | 3 |
| 11RD | 6 | 6 | 4 | 3 | 5 | 4 | 3 | 3 | 3 | 2 | 2 | 2 |
| 12MZ | 6 | 5 | 4 | 4 | 6 | 5 | 4 | 4 | 4 | 4 | 2 | 2 |
| 13OP | 3 | 3 | 2 | 2 | 4 | 3 | 3 | 2 | 4 | 3 | 2 | 2 |
| 14DT | 5 | 5 | 4 | 3 | 5 | 4 | 3 | 2 | 4 | 4 | 3 | 3 |
| 15CS | 4 | 4 | 4 | 2 | 4 | 3 | 3 | 2 | 4 | 3 | 3 | 2 |
| 16LM | 5 | 5 | 4 | 4 | 5 | 4 | 3 | 3 | 4 | 4 | 3 | 3 |
| 17EFC | 6 | 5 | 4 | 3 | 5 | 4 | 3 | 3 | 4 | 4 | 3 | 3 |
| 18CCO | 7 | 7 | 6 | 5 | 5 | 4 | 4 | 3 | 3 | 2 | 2 | 2 |
| 19EP | 7 | 6 | 6 | 5 | 6 | 6 | 5 | 4 | 4 | 4 | 2 | 2 |
| 20AMA | 5 | 5 | 4 | 3 | 4 | 4 | 4 | 3 | 4 | 3 | 3 | 3 |
| 21PL | 6 | 6 | 5 | 3 | 5 | 4 | 3 | 3 | 4 | 4 | 4 | 3 |
| 22CA | 5 | 4 | 4 | 3 | 4 | 3 | 3 | 3 | 4 | 4 | 3 | 2 |
| 23BTC | 6 | 5 | 5 | 4 | 5 | 4 | 4 | 3 | 4 | 4 | 4 | 3 |
| 24LB | 5 | 4 | 2 | 2 | 5 | 4 | 2 | 2 | 5 | 5 | 4 | 4 |
| 25GN | 4 | 3 | 3 | 2 | 3 | 3 | 3 | 2 | 4 | 4 | 3 | 3 |
| MEDIA | 5.3 | 4.8 | 3.7 | 3.0 | 4.7 | 4.0 | 3.1 | 2.7 | 4.0 | 3.5 | 2.8 | 2.4 |

LEFT THIGH THERMOGRAPHY

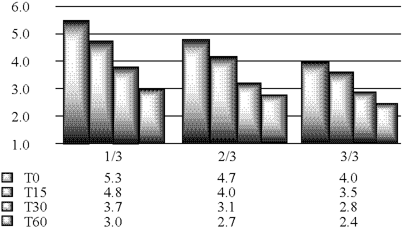

1. Carnitine salt, characterised in that it consists of carnitine hydroxy citrate of formula 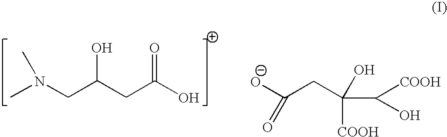
of natural origin.

2. Salt according to claim 1, characterised in that it is obtained from the reaction of 3-carboxy-2-hydroxy-propyl trimethyl amine of formula 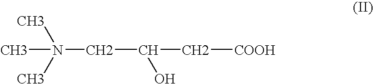
with L-hydroxy citric acid of formula

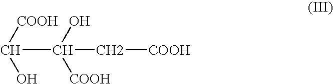

3. Salt according to claim 2, characterised in that said L-hydroxy citric acid is extracted from the juice of fruits of the botanical species known as “Garcinia Cambogia”.

4. Preliposome for preparing dermo-cosmetic formulae for topical use, characterised in that it contains carnitine hydroxy citrate according to one or more of the previous claims.

5. Preliposome according to claim 4, characterised in that it also contains pure phosphatidylcholine and xantana rubber.

6. Dermo-cosmetic formula for topical use, characterised in that it is prepared with the preliposome according to one or more of the previous claims.

7. Formula according to claim 6, characterised in that it is in the form of a cosmetic gel.

8. Formula according to claim 7, characterised in that it has the following formulation:

| | |
|---|---|
| Hydroxy ethyl cellulose | 1.8 g |
| Triethanol amine | 0.9 g |
| Solubiliser, fragrance, preservatives | 2.5 g |
| Preliposome | 13 g |

9. Formula according to claim 7, characterised in that it is in the form of emulsion, lotion, or other similar types of cosmetics.

10. Use of carnitine salt and of the formula according to one or more of the previous claims for treating skin blemishes linked to physiological disorders ascribable to cellulite and anomalous adiposity connected to this.

11. Use of carnitine salt and of the formula according to one or more of the previous claims as a food supplement and the like.
